(19) United States
(12) Plant Patent Application Publication
(10) Pub. No.: US 2017/0367246 P1
(43) Pub. Date: Dec. 21, 2017

(54) MONARDA PLANT NAMED 'COTTON CANDY'

(50) ;

(71) Applicant: Hans A. Hansen, Zeeland, MI (US)

(72) Inventor: Hans A. Hansen, Zeeland, MI (US)

(73) Assignee: Walters Gardens, Inc., Zeeland, MI (US)

(21) Appl. No.: 14/999,735

(22) Filed: Jun. 21, 2016

(51) Int. Cl. A01H 5/02

(52) U.S. Cl. CPC A01H 5/02
USPC PLT/455

(57) ABSTRACT

The new and distinct cultivar of ornamental cultivar of bee balm plant named Monarda ‘Cotton Candy’ is a short, winter-hardy, compact, numerous-stemmed well-branched, densely clumping habit with dark green foliage numerous early-flowering lilac-pink-colored flowers creating excellent flower coverage over a prolonged period of time. ‘Cotton Candy’ has strong foliar resistance to powdery mildew.

Botanical classification: Monarda hybrid.

Variety denomination: ‘Cotton Candy’.

### BACKGROUND OF THE INVENTION

The present invention relates to the new and distinct cultivar of bee balm plant, botanically known as Monarda ‘Cotton Candy’, and hereinafter also referred to solely as the cultivar ‘Cotton Candy’ or the “new plant”. The new plant was selected from seed collected by the hybridizer from a formal hybridizing program on Aug. 9, 2012 from designated hybridizing plot of a wholesale perennial nursery in Zeeland, Mich., US. The seed parent was an unreleased proprietary hybrid identified as H10-26-09 (not patented) that was allowed to be insect pollinated in an isolation block on Jun. 12, 2012, and the seeds were harvested on Aug. 9, 2012. The new plant was a single seedling selection originally evaluated in the summer of 2013 from the all the seeds that were sown in the fall of 2012 from this specific parent process. Further selection was completed in the trial fields of a nursery in Zeeland, Mich. in the summer of 2014 and the plant was assigned the breeder code of H12-59-3. The plant has been asexually propagated by shoot tip cuttings originally in 2014 in greenhouses at the same nursery in Zeeland, Mich., and the subsequent asexually propagated plants found to be stable, true to type and identical to the original selection.

No plants of Monarda ‘Cotton Candy’ have been sold in this country, or anywhere in the world, prior to the filing of this application, nor has any disclosure of the new plant been made prior to the filing of this application with the exception of that which was disclosed within one year of the filing of this application and was either derived directly or indirectly from the inventors.

### BRIEF SUMMARY OF THE PLANT

Monarda ‘Cotton Candy’ is unique from its parents and all other bee balm plants known to the inventors. It is a complex hybrid probably containing at least the species M. didyma, M. bradburiana, M. bartlettii and M. fistulosa. The nearest comparison varieties known to the applicant are: ‘Pete's Purple’ (not patented), ‘Leading Lady Lilac’ U.S. Plant Pat. No. 26,431 and ‘Leading Lady Plum’ U.S. Plant Pat. No. 26,447. Compared to ‘Pete's Purple’, the new plant has lighter colored flower petals of a lilac-pink hue rather than purple, and the new plant is significantly shorter in habit. The new plant is slightly taller than both ‘Leading Lady Lilac’ and ‘Leading Lady Plum’ and the flower color of the new plant is lilac-pink compared to the light lilac and magenta purple flower colors, respectively. ‘Leading Lady Lilac’, ‘Leading Lady Plum’ and ‘Cotton Candy’ all have darker spotting on the inside flower petals, however ‘Pete's Purple’ does not have spotting on the flower petals.

The following are traits of Monarda ‘Cotton Candy’ that in combination distinguish it from all other bee balm known to the inventor:
1. Short, winter-hardy, compact, well-branched, densely clumping habit.
2. Medium to dark-green, lemony-fragrant leaves.
3. Strong powdery mildew resistant foliage.
4. Medium-sized, lilac-pink-colored flowers for a long period in summer with excellent flower coverage.

### BRIEF DESCRIPTION OF THE DRAWINGS

The photographs of the new plant demonstrate the unique traits of ‘Cotton Candy’ and the overall appearance of the plant at two-years old in the full sun trial garden of a nursery in Zeeland, Mich. The colors are as accurate as reasonably possible with color reproductions. Variation in ambient light spectrum, source and direction may cause the appearance of minor variation in color.

FIG. 1 shows the new plant in flower in the landscape.

FIG. 2 shows a close-up of the flowers.

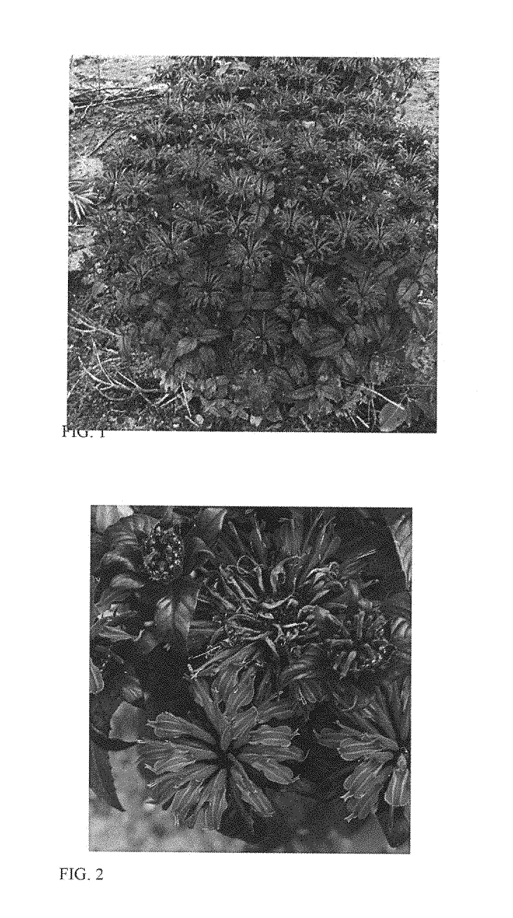

### DETAILED BOTANICAL DESCRIPTION

The following descriptions and color references are based on the 2001 edition of The Royal Horticultural Society Colour Chart except where common dictionary terms are used. Monarda ‘Cotton Candy’ has not been observed under all possible environments. The phenotype may vary slightly with different growing environments such as temperature, light, fertility, soil pH, moisture and maturity levels, but without any change in the genotype. The following observations and size descriptions are based on two-year old greenhouse-grown plants in gallon containers at a nursery in Zeeland, Mich. with supplemental fertilizer and water as needed but without plant growth regulators or pinching.
Botanical classification: Monarda hybrid;
Parentage: Female (seed) parent is H10-26-09 (not patented); male (pollen) parent is unknown;
Plant habit: Winter-hardy herbaceous perennial, compact, producing several stems spreading by short rhizomes; about 35 cm tall at flowering and about 50 cm wide; flowering begins early-summer in Michigan and continuing for about 5 to 8 weeks;
Propagation: Stem cuttings;
Time to produce finished crop in 3.8 liter pots: About 10 to 12 weeks; moderate rate of growth;
Root: Fine, fibrous and freely branching; color creamy white to tan depending on soil type;
Leaves: Simple, lanceolate, opposite, serrated, puberulent above and below; lustrous above, initial matte below becoming slightly lustrous; acute apex, rounded to cordate base; to about 8.7 cm long by about 3.5 cm wide, average about 7.2 cm long and 2.8 cm wide;
Leaf color: Young expanding leaves between RHS 144A and RHS 143A on adaxial surface, abaxial surface expanding leaves between RHS 144A and RHS 144B; mature adaxial leaves darker than RHS 139A and abaxial leaves nearest RHS 137A;
Foliage fragrance: Pleasant, lemony;
Veins: Pinnate; Puberulent abaxial, minutely puberulent adaxial, slightly sunken above and raised below;
Vein color: Mature leaves adaxial midrib and lateral veins nearest RHS 144C; mature abaxial midrib and primary veins nearest RHS 139D, secondary abaxial veins nearest RHS 138B; emerging leaves adaxial midrib and lateral veins nearest RHS 145C; emerging leaves abaxial midrib nearest RHS 145C, lateral veins on emerging primary abaxial leaves between RHS 145B and RHS 146D with slight blush of nearest RHS 187A;
Bracts: Between distal leaves and flowers; sessile; lanceolate, with acuminate apex and cuneate base; puberulent abaxial and slightly puberulent adaxial; lowest bracts about average about 4.3 cm long and 1.6 cm wide decreasing distally to about 1.0 cm long and 1.0 mm wide;
Bract color: Abaxial and adaxial main color nearest RHS 139A with tinting of nearest RHS N186C; and proximal bract adaxial veins nearest RHS 186B and abaxial nearest RHS 145C; distal bracts nearest RHS 145A abaxial and adaxial with midrib and lateral veins also RHS 145A;
Petiole: Pubescent, applanate adaxial, rounded abaxial; average about 2.0 mm long and 2.0 mm across;
Petiole color: Nearest RHS 138B adaxial and between RHS 138B and RHS 139D abaxial;
Stems: Quadrangular, sparsely puberulent; densely puberulent at nodes; about 4.0 mm across at base and about 33 cm long;
Stem color: Nearest RHS 141A distally and nearest RHS 146D proximally;
Nodes: About 12 per stem; average internode about 2.5 cm; color same as surrounding stem;
Branches: Mostly in upper nodes as flowers dehisce or are removed;
Flowers: Single, labiate, actinomorphic flowers arranged in dense terminal verticillate head about. 8.0 cm across and 1.9 cm tall opening from the center and progressing outwardly and down; each flower persisting about 5 days in Michigan; numerous, about 280 flowers per terminal verticil and about 80 to 100 in lateral verticils; flower altitude upright to slightly outward in center of verticil to horizontal toward outer perimeter of verticil; self-cleaning;
Flower period: Usually late June into August; about six to seven weeks;
Flower fragrance: Moderately sweet;
Buds one to two days prior to opening: Narrowly oblanceolate with terete base, applanate laterally; tubular base; rounded apex; arcuate slightly downward in distal half; about 2.5 cm long and about 4.8 mm tall in the middle and 3.0 mm across;
Bud color one to two days prior to opening: Nearest RHS N74D in distal one-half, white base, lighter than RHS N155D or RHS 155D;
Petals: Labiate, arcuate downward; fused in about the basal 2.0 cm; split in two in about the distal 1.3 cm; upper lip fused into a conduplicate hood about 3.3 cm long and 2.5 mm across at distal fusion point of two lips; lower lip about 3.5 cm long and about 8.0 mm across, comprising three fused lobes including two shorter side lobes split in the terminal 1.0 mm, side lobes with rounded apex, center lobe about 1.0 mm across and about 4.0 mm with emarginate apex about 0.5 mm deep; both lobes glandular and puberulent outer surfaces with hairs the same color as petals; both lips glabrous on inner surfaces;
Petal color: Basal 8.0 mm white, lighter than RHS 155D; abaxial distal upper lip nearest RES N74C, adaxial distal upper lip between RHS N74D and RHS 75C in longitudinal center with margin region lighter than RHS 69D; lower lip about 8.0 mm adaxial base nearest white, lighter than RHS 155D, distal two-thirds abaxial surface nearest RHS N74C with distal one-third edges lightening to nearest RHS 69C, adaxial distal region nearest RHS 75C with spots of nearest RHS 72B;
Androecium: Two;
   Filaments.—Two, about 7.0 mm long and 0.5 mm diameter; basally fused to lower lip; color distally nearest RHS 69C and basally white, lighter than RHS 155D.
   Anther.—One; at terminally fused filaments; oblong elliptic, dorsifixed, longitudinal; 2.2 mm by 1.0 mm; color nearest RHS 70A prior to dehiscing, after dehiscing nearest RHS N186A.
   Pollen.—Abundant, elliptic to globose, less than 0.1 mm; color nearest RHS 11D;
Gynoecium: one per flower.
   Style.—About 3.9 cm long by about 0.5 mm diameter; slightly arcuate downward; color basal 2.5 cm white, lighter than RHS 155D and distally nearest RHS 77B.
   Stigma.—Split in two in the distal 2.0 mm, 0.25 mm in diameter; color nearest RHS 77B.
   Ovary.—Four seeded; 1.0 mm by 0.75 mm; color nearest RHS N144D.
   Fruit.—Up to four per flower; nutlet; slightly applanate ellipsoid; about 1.2 mm long and 0.8 mm across and 0.7 mm thick; color blend between RHS 164B and RHS 199C.
Calyx: five fused sepals, apiculate apex, base fused forming corolla about 13.0 mm long and 2.0 mm diameter with apical 2.0 mm separated; adaxial apex glandular, abaxial puberulent;
Sepal color: Abaxial base translucent to nearest RHS 145D, mid-section between RHS 145C and 138A, apex nearest RHS N186A; adaxial base translucent to RHS 145D, mid-section nearest RHS 145C with veins of nearest RHS 138A, apex nearest RHS N186A;
Peduncle: Pubescent, stiff, strong, erect, quadrangular to about 0.4 cm across and average about 29.0 cm long; about 70 per plant; naturally branched at nodes;
Peduncle color: Nearest RHS 141A distally and nearest RHS 146D proximally;
Pedicel: Terete; about 0.5 mm long and 0.5 mm diameter; color nearest RHS 144A;
Hardiness: The new plant grows best with plenty of moisture and adequate drainage; hardy to at least from USDA zone 4 through 9;
Disease and pest resistance: Demonstrated strong powdery mildew resistance. No resistance beyond that typical of other bee balms has been noted.

I claim:

1. A new and distinct cultivar of ornamental bee balm plant, Monarda ‘Cotton Candy’, as herein described and illustrated.
